(19) United States
(12) Patent Application Publication
(10) Pub. No.: US 2015/0272813 A1
(43) Pub. Date: Oct. 1, 2015

(54) SYSTEMS AND METHODS FOR ELECTRICAL STIMULATION OF THE GASTROINTESTINAL TRACT FOR TREATMENT OF POST-OPERATIVE ILEUS

(71) Applicant: Regents of the University of Minnesota, Minneapolis, MN (US)

(72) Inventors: Laura PAULSEN, Cambridge, MA (US); James KROCAK, Minneapolis, MN (US); Jesus CABRERA, Minneapolis, MN (US); Raed RIZQ, Maple Grove, MN (US); Badrinath KONETY, Edina, MN (US); Saurav PAUL, Minneapolis, MN (US)

(21) Appl. No.: 14/672,541

(22) Filed: Mar. 30, 2015

Related U.S. Application Data
(60) Provisional application No. 61/973,005, filed on Mar. 31, 2014.

(51) Int. Cl. A61H 9/00; A61M 1/00; A61N 1/36; A61N 1/05; A61M 25/10

(52) U.S. Cl. CPC A61H 9/0007; A61N 1/0509; A61M 25/10; A61N 1/36007; A61M 1/008; A61M 2025/105; A61M 2230/005; A61M 2230/63; A61M 2230/20; A61M 2230/08; A61M 2210/1042; A61H 2201/0103

(57) ABSTRACT

The present disclosure is directed to a medical instrument. Systems and methods are provided for stimulation of the gastrointestinal tract. The medical instrument may include an elongate component having a proximal end and a distal end. The medical instrument may be configured for insertion in a natural orifice of a patient and to traverse the gastrointestinal tract of the patient. The medical instrument may include a handle at the proximal end and a stimulator at the distal end wherein the stimulator may be configured to stimulate the gastrointestinal tract to effect coordination of peristaltic waves.

### CROSS-REFERENCE TO RELATED APPLICATIONS

This application claims the benefit of priority from U.S. Provisional Application No. 61/973,005, filed on Mar. 31, 2014, which is incorporated by reference herein in its entirety.

### DESCRIPTION OF THE EMBODIMENTS

### Technical Field

Embodiments of the present disclosure relate generally to medical instruments. More particularly, embodiments of the disclosure relate to medical instruments for use in medical applications, such as, for example, stimulation of the gastrointestinal (GI) tract for treatment of postoperative ileus (POI). Embodiments of the disclosure also cover methods of using such instruments.

### BACKGROUND OF THE DISCLOSURE

Postoperative ileus (POI) is the transient impairment of intestinal motility occurring after a surgical procedure. This is clinically characterized by abdominal pain and distress, abdominal distention, delayed passage of gas and stool, lack of bowel sounds, and accumulation of gas and fluid in the bowel that may result in nausea, pain, and vomiting. POI is a major health care problem that adversely influences many aspects of postoperative patient care including overall prolonged recovery, increased morbidity, and extended hospitalization.

The exact pathophysiologic basis of POI is unknown, although it is believed to be multifactorial and includes three major mechanisms; neurogenic, inflammatory, and pharmacologic. POI normally resolves within four days following an abdominal surgical procedure but may range from not being present and/or as little as two days following laparoscopic surgery to more than 9 days after major abdominal surgeries, including laparotomies. The length of hospitalization following surgery depends on several factors such as surgical procedure, postoperative pain, patient's co-morbidities, and the duration of POI.

POI increases the utilization of hospital resources because discharge after surgery is typically delayed until the patient can tolerate a regular diet and acceptably healthy bowel function is restored. Data from the Health Care Financing Administration (HCFA) showed that in 2000, a total of 161,000 Medicare patients underwent major abdominal colorectal procedures, stayed in the hospital 1.82 million days (a mean of 11.3 days per patient), and cost approximately $1.75 billion.

Goldstein et al. found, in an article evaluating the inpatient economic burden of POI associated with abdominal surgery, that a hospital stay coded POI is both substantially more costly ($18,877 vs. $9,460 per case) and of longer duration (11.5 days vs. 5.5 days per case). (Goldstein J L, Matuszewski K A, Delaney C P, Senagore A, Chiao E F, Shah M, Meyer K, Bramley T. Inpatient economic burdern of postoperative ileus associated with abdominal surgery in the United States. P&T 2007; 32(2):82-90.) This study documented that the total annual United States hospital cost attributed to managing cases coded POI is $1.46 billion for both the index hospitalization and any readmissions within 30 days.

Current management strategies consist of careful selection of anesthesia and analgesia before, during, and after surgery to minimize the duration of POI and the use of supportive therapies such as enteral nutrition, intravenous fluids and pharmacological agents (i.e., laxatives and prokinetic drugs). However, these strategies are not uniformly successful and patients are often slow to respond to them.

For example, the supportive therapies listed above, including pharmacological agents, may be controlled only before the provided nutrition fluid, or agent enters the body. In some cases, a healthcare practitioner may not, once the supportive therapy is ingested, be able to make adjustments based on, for example, patient reaction or effectiveness. Further, many of these conventional solutions must be prescribed to all pre-operative patients who may or may not develop POI, without the ability to select and treat only those patients that actually suffer from POI. As such, there exists a need for improved medical instruments and procedures for treatment of POI.

### SUMMARY OF THE DISCLOSURE

Embodiments of the present disclosure provide systems and methods for stimulation of the gastrointestinal tract for treatment of post-operative ileus.

One embodiment of the present disclosure is directed to a medical instrument. The medical instrument may include an elongate component having a proximal end and a distal end and may be configured for insertion in a natural orifice of a patient and to traverse the gastrointestinal tract of the patient. The medical instrument may also include a handle at the proximal end; and a stimulator at the distal end, wherein the stimulator is configured to stimulate the gastrointestinal tract to effect coordination of contractile peristaltic waves.

In various embodiments, the medical instrument may include one or more of the following features: wherein the stimulator provides at least one of electrical, mechanical, and enteric stimulation; wherein the stimulator is configured to be in a collapsed state for insertion into the gastrointestinal tract, and wherein the stimulator is configured to expand from the collapsed state to a deployed state to contact an interior surface of the gastrointestinal tract; wherein the distal end of the tubular component includes at least one port for at least one of delivering food, delivering pharmacologic agents, removing waste, and removing gas; including a control unit configured to produce patterns of stimulation to the stimulator based on at least one of real-time user input and pre-set stimulation patterns; including a sensor for sensing at least one parameter of motion, stress, strain, contact impedance, electrical signals, and chemical biomarkers; wherein the control unit is configured to adjust stimulation provided by the stimulator based on the at least one parameter sensed by the sensor; wherein the distal end is positioned based on the at least one parameter; wherein the stimulator is expanded by inflation; wherein the stimulator is expanded via a pull-wire or pulley system; wherein the interior surface of the gastrointestinal tract is a portion of the small intestine of the patient; including a protective sheath; wherein the stimulator is configured to stimulate the gastrointestinal tract in a pattern of repeating stimulation; wherein the pattern comprises a stimulation of between 0.5V and 1.5V; and/or repeating the pattern for stimulation until sustained coordinated peristaltic waves are sensed.

Another embodiment of the present disclosure is directed to a method for treatment of a gastrointestinal tract. The method may include positioning a distal portion of a medical instrument, including a stimulator configured to stimulate tissue, at a target region within the gastrointestinal tract; and stimulating tissue of the target region, via the stimulator, to effect coordination of peristaltic waves. The method may also include producing patterns of stimulation based on at least one of real-time user input and pre-set stimulation patterns; deploying the stimulator to be in physical or electrical communication with tissue of the target region; wherein the stimulator is deployed by expanding the stimulator; wherein the stimulator is deployed by inflation; wherein the stimulator is deployed via a pull-wire or pulley system; wherein stimulating tissue includes at least one of electrical, mechanical, and enteric stimulation; sensing a location of a contraction and selecting the target region based on the location of the contraction; sensing a parameter within the patient's body and basing a pattern for stimulating the target region on the sensed parameter; repeating the pattern for stimulation until sustained coordinated peristaltic waves are sensed stimulating the target region by delivering electrical pulses; protecting the stimulator of the medical instrument via a sheath; and/or wherein the target region within a gastrointestinal tract is a region of the small intestine.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the scope of disclosed embodiments, as set forth by the claims.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 illustrates an exemplary embodiment of a tubular component of a medical instrument inserted into a patient's natural body orifice;

FIG. 2 illustrates an exemplary embodiment of the tubular component including a proximal end, a variable length of tubing, and a distal end;

FIG. 3 illustrates an exemplary embodiment of a handle portion connected to the proximal end of the tubular component;

FIG. 4 illustrates an exemplary embodiment of the distal end of the tubular component, including a port, sensors, and a distal assembly;

FIGS. 5A and 5B illustrate a distal portion of the tubular component including an alternative embodiment of the distal assembly in its collapsed and deployed configurations;

FIGS. 6A and 6B illustrate a distal portion of the tubular component including an alternative embodiment of the distal assembly in its collapsed and deployed configurations;

FIGS. 7A and 7B illustrate a distal portion of the tubular component including an alternative embodiment of the distal assembly in its collapsed and deployed configurations;

FIGS. 8A and 8B illustrate a distal portion of the tubular component including an alternative embodiment of the distal assembly in its collapsed and deployed configurations;

FIG. 9 illustrates an alternative embodiment of a control unit including a delivery port and a removal port; and

FIG. 10 is a block diagram of an exemplary method of using medical devices disclosed herein.

### DESCRIPTION OF THE EMBODIMENTS

Reference will now be made in detail to exemplary embodiments of the disclosure, examples of which are illustrated in the accompanying drawings. Wherever possible, the same reference numbers will be used throughout the drawings to refer to the same or like parts.

Embodiments of the present disclosure relate to systems and methods for treatment of POI. The medical device described herein works by stimulation of the GI tract. More specifically, in exemplary embodiments, the myenteric plexus and/or submuscosal plexus may be stimulated in order to promote resolution of POI through re-coordination of contractile peristaltic waves. A segment of gastrointestinal tract of only less than two inches may need to be stimulated in order to initiate a cascade effect on additional gastric pacemaker cells. The stimulation could be axial in nature, circumferential, or a combination thereof. Further, sustained stimulation over multiple hours may result in propagation of the stimulation effect to the distal parts of the GI tract, including the colon, and proximal parts, including the stomach.

Unlike a pill or other pharmacological agents used for conventional solutions, devices and methods of the present disclosure can be used post-operatively on only those patients that are affected with POI, as opposed to being prescribed to all pre-operative patients. Further, this internal device for electrically stimulating the GI tract may be externally controlled, as opposed to medication that, once ingested, may no longer be adjusted by a medical professional. Further, devices and methods of the present disclosure provide direct or very close electrical stimulation to the target region of the GI tract, as opposed to surface electrical stimulation which has to traverse layers of soft tissue and may not be specific to a targeted region.

A. The Tubular Component

The medical device of the present disclosure may include a control unit and a tubular component. The tubular component may be comprised of three main components: (1) a variable length of tubing; (2) a handle, at the component's proximal end, for externally manipulating the tubular structure into, inside of, and out of the patient; and (3) a distal assembly at the distal portion of the tubular component.

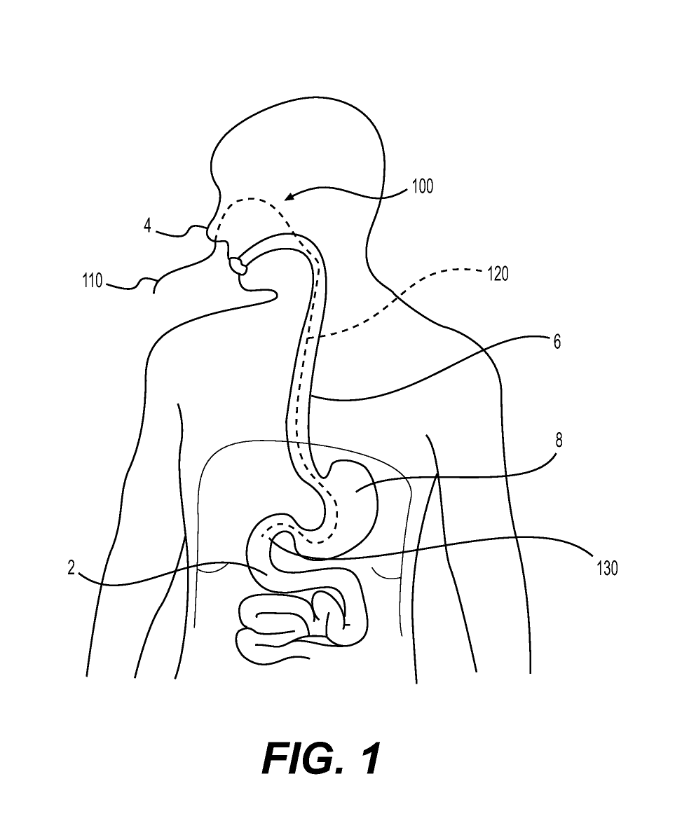

FIG. 1 depicts the tubular component 100 according to an exemplary embodiment of the disclosure. Tubular component 100 may be configured for insertion into a patient's body through an anatomical opening. Accordingly, tubular component 100 may be shaped and sized for placement into a patient via a body orifice or an incision.

In some embodiments, tubular component 100 may be inserted and extracted from the patient through the mouth or through the nasal canal 4, as depicted in FIG. 1. Both approaches will enable the user to insert and extract the device through the GI tract including, but not limited to, the esophagus 6, stomach 8, and the small intestine 2. Additionally, tubular component 100 may be configured for a transrectal approach. Tubular component 100 may include, at least, a proximal end 110, a distal end 130, and lumen 122 extending the length of tubular component 100 between proximal end 110 and distal end 130. Tubular component 100 can have any suitable length sufficient to reach the treatment site within a patient. The length of the tubular component 100 may be sufficient so that the proximal end 110 is external to the patient's body and the distal end 130 is internal to the patient's body, e.g. within the small intestine 2. FIG. 1 depicts the tubular component 100 extending through an anatomical opening (depicted as the nose in FIG. 1), through the esophagus 6, stomach 8, and into the small intestine 2. In some embodiments, the distal end 130 may be in contact with the proximal small intestine lumen 2. In other embodiments, the distal end 130 may be within the jejunum of the small intestine 2. For example, the tubular component 100 may be configured in a similar manner to a nasojejunal (NJ) tube and may be inserted through the nasal passage and into the jejunum.

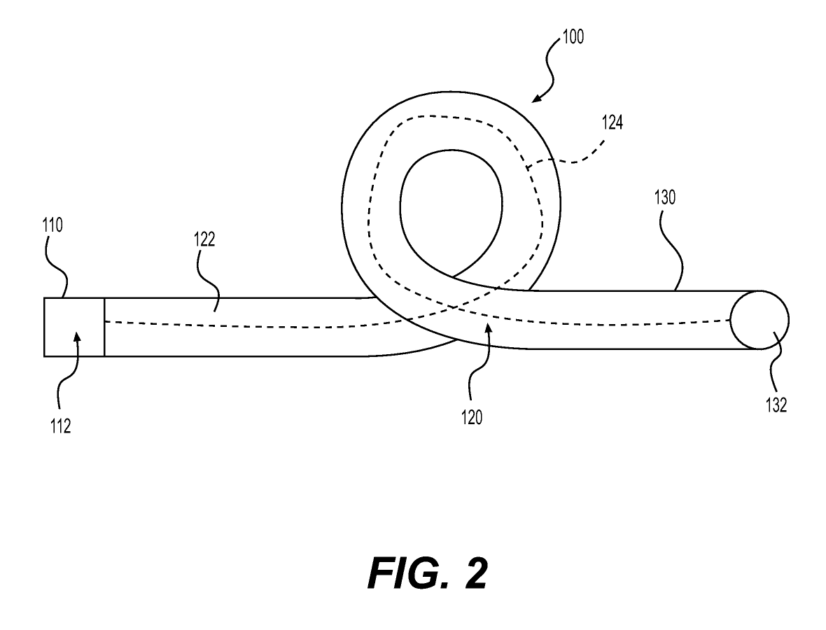

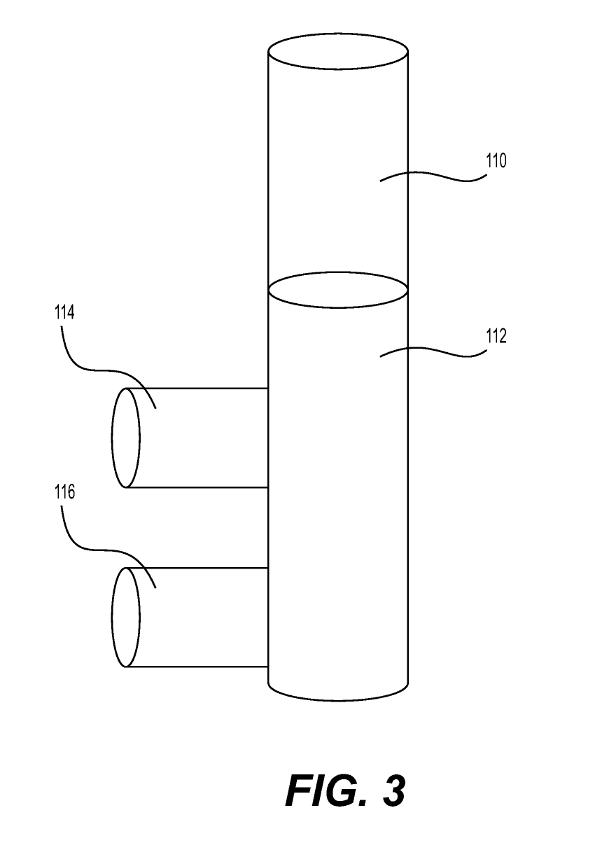

FIG. 2 shows an exemplary embodiment of tubular component 100. Tubular component 100 may include, at least, three main components: (1) a variable length of tubing 120; (2) a handle portion 112 for externally manipulating the tubular component into, inside of, and out of the patient as further depicted in FIG. 3; and (3) a distal assembly 132 at the distal portion 130 of the tubular component.

1. The Tubing

The variable length of tubing 120 extends between the proximal end 110 and the distal end 130. The length of tubing 120 may vary depending upon patient need. The length of the tubular component may be sufficient so that the proximal end 110 is external to the patient's body and the distal end 130 is internal to the patient's body. In certain embodiments, the distal end 130 extends through an anatomical opening (i.e. the patient's mouth or nose) and may be in contact with the interior wall of the small intestine.

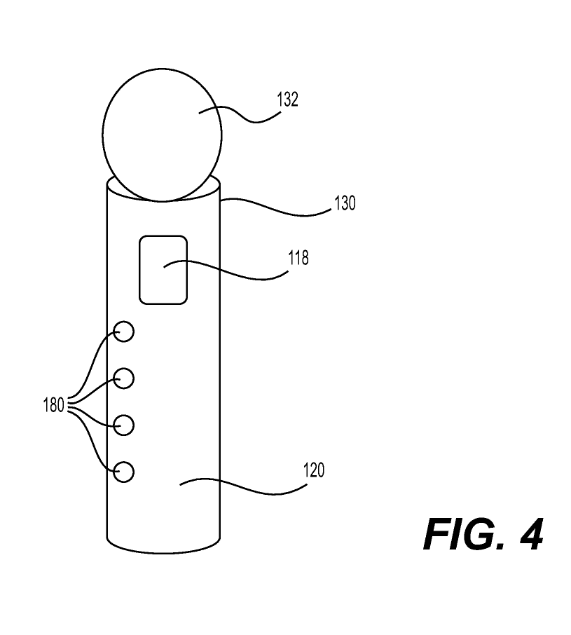

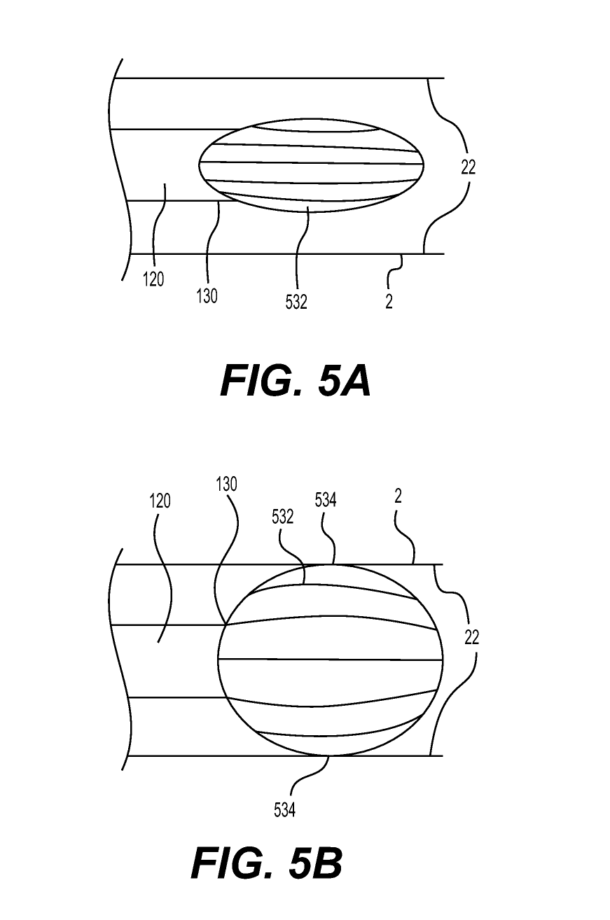

Tubing 120 may be attached to the handle portion 112 at the proximal end 110 and at distal end 130 may be attached to a variety of components and mechanisms including, but not limited to, distal assembly 132 that may provide stimulation in the GI tract, as depicted in FIGS. 5-8, and components that may deliver food and/or medicine and remove gases as depicted in FIG. 4.

In some embodiments, the tubing 120 may be comprised of a hollow cylindrical structure wherein a lumen 122 extends from proximal end 110 to distal end 130. The tubing 120 may include components embedded within the lumen 122, including, but not limited to, (i) a wire 124, as depicted in FIG. 2, to conduct the electricity from the proximal end 110 at the handle portion 112 to the distal end 130; and (ii) a retraction mechanism, as will be further described below, that may deploy and retract a stimulation mechanism.

The diameter of tubing 120 may be selected based on the desired application, with the largest diameter of tubing 120 generally chosen to be smaller than the typical diameter of the desired body lumen where tubing 120 may be used. Tubing 120 to be employed in the esophagus, for example, may be smaller than tubing to be employed in the colon. Those of ordinary skill will recognize that the diameter (or any other dimension) of tubing 120 may also depend on the insertion location of tubing 120. That is, if tubing 120 is desired to be used in a patient's colon but is intended to be inserted into a patient via the patient's nose, the diameter of tubing 120 may be selected to be smaller than a nasal passage of the tubing, for example. In one embodiment, tubing 120 may be a tubular structure. This structure may have a substantially circular cross-section or an elliptical, oval, polygonal, or irregular cross-section may be employed, as desired. In addition, a select portion of tubing 120, such as, e.g., a distal portion, may have a cross-sectional configuration or dimension different from another portion, e.g., a proximal portion, of tubing 120. Moreover, tubing 120 may be flexible along its entire length or adapted for flexure along portions of its length. Alternatively, the distal end of tubing 120 may be flexible while the remainder of tubing 120 may be semi-rigid or otherwise relatively less flexible. Flexibility allows tubing 120 to maneuver turns in body lumens, while some level of rigidity provides a structure upon which the operator can exert the necessary force to urge tubing 120 forward. Tubing 120 may be made of any suitable biocompatible material such as a polymeric, metallic, or rubber material. Tubing 120, or a portion thereof, may be also made from a malleable material, such as stainless steel or aluminum, allowing a physician to change the shape of tubing 120 before or during a procedure. In some instances, tubing 120 may be composed of an extrusion of wire braided polymer material to impart flexibility. Tubing 120 may also be coated using suitable low friction material, such as TEFLON®, polyetheretherketone (PEEK), polyimide, nylon, polyethylene, or other lubricious polymer coatings, to reduce surface friction with the surrounding body tissues. Additionally or alternatively, one or portions of the surfaces of tubing 120 may be coated or otherwise covered with a non-conductive material for preventing short-circuiting between tubing 120 and electrically conductive elements, such as the electrodes described in more detail below.

2. The Handle Portion

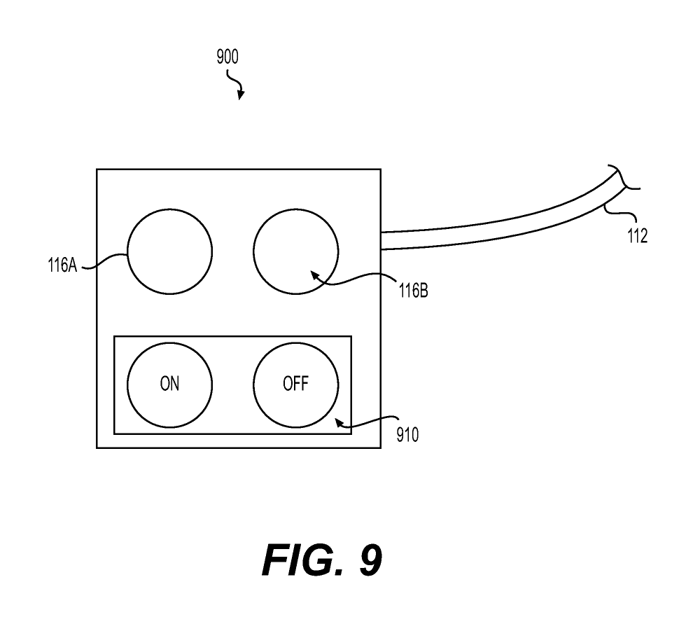

Handle portion 112 is disposed at the proximal end 110 of the tubular component 100. Handle portion 112 may be any known, suitable handle. Handle portion 112 may be externally manipulated by the user to facilitate entry and removal of the other attached device components to be inserted into, stay in, and be removed from the patient. As illustrated in FIG. 3, handle portion 112 may include an electricity port 114 that may be removably attached to an electrical source (not shown) and may allow delivery of electrical energy, signals, and/or light to distal end 130. Electricity may travel through electricity port 114 and wire 124, as depicted in FIG. 2, to provide stimulation of tissue in contact with the distal end 130. Wire 124 may be insulated and/or placed in the wall of the tubing to protect it from mechanical damage and/or digestive fluids. Handle portion 112 may additionally include a delivery and removal port 116 for allowing delivery of, for example, food, fluid and/or medication to the distal end 130 and/or the removal of waste and/or gas from distal end 130. Electricity port 114 and delivery/removal port 116 may connect to a control unit 900 as depicted in FIG. 9, as will be further described below.

It should be noted that the functions performed by electricity port 114 and delivery/removal port 116, as described herein, may be implemented using a single port. It should also be noted that that the functions performed by electricity port 114 and/or delivery/removal port 116, as described herein, may be implemented using multiple ports.

Further, in an alternative embodiment, the operation of the device and, in particular, the mechanisms disposed at the distal end 130 may be operated wirelessly. In that embodiment, at least certain functions performance by electricity port 114 may not be necessary.

3. The Distal End

The distal end 130 of the tubular component 100 may be comprised of one or more of a sensing mechanism, an exchange mechanism, a retraction mechanism, a positioning mechanism, a protective mechanism, and/or a stimulation mechanism, all described below. It should be noted that any of the functions performed by each of these mechanisms, as described herein, may be implemented by a single mechanism or a combination of mechanisms.

The medical device may include a sensing mechanism. The sensing mechanism may be configured to sense multiple key parameters such as, but not, limited to, motion, stress, strain, contact impedance, electrical signals, and/or chemical biomarkers. The sensing mechanism may include a plurality of sensors 180 of FIG. 4 at multiple locations along the length and circumference of the medical device. The programmed system and/or a control unit may be configured to utilize the data regarding these or other parameters to actively control or optimize the stimulation parameters and/or patterns. For example, the data from the sensing mechanism may be sent to the control unit to more optimally control the tissue stimulation in order to influence the contractions and/or relaxations in the intestinal smooth muscle cells.

The sensing mechanism may also be used to help position the medical device, including the distal end 130 of the tubular component 100. The sensing mechanism may sense at multiple locations along the length and circumference of the tubular component, contractions in the small intestine via impedance, stress, and/or strain or other modalities. The location of contractions may be used to ensure contact with a desired target region. In another embodiment, the distal end 130 may include indicia, visible under various imaging regimes. For example, radiopaque or sonoreflective markings (not shown) may be added to an exterior surface of the distal end 130 or distal assembly 132 to indicate position and orientation during a procedure. That information can enable the user to track the medical device, ensure contact with the target region, and avoid potential damage to sensitive tissues.

In one embodiment, the distal end 130 may also include a protective mechanism that may be configured to protect the stimulation mechanism and/or other components (e.g. pharmacologic agents) from mechanical damage and/or digestive fluids. For example, the protective mechanism may serve to prevent scratching along the nasal-jejunal passage and/or prevent digestive enzymes from inactivating the pharmacologic agent(s) that may exist on the device for delivery at the treatment site. The protective mechanism may include, but is not limited to, an insulated tube or a sheath-like mechanism that can be placed in the wall of tube 120 or within lumen 122 of tube 120. In some embodiments, the protective mechanism may assist in re-capturing and re-positioning the stimulation mechanism.

A sheath from the protective mechanism or a separate sheath may be used to steer and position the tubular component 100. This positioning sheath may be on the exterior of the distal end 130 of the tubular component 100 and adapted to be moved into a body lumen. As known in the art, the positioning sheath may be fitted with steering capability, actuated by control wires or rods. Steering devices are well known in the art and will not be described further here.

The distal end 130 of the tubular component 100 may also, or alternatively, include a retraction mechanism to deploy and retract stimulators of the medical device, at the targeted region within the GI tract. The retraction mechanism may include a pulley to deploy and retract the stimulation mechanism. For example, a pull wire may have one end attached to the distal assembly (e.g. stimulation mechanism) and another end attached to the handle. Extension and retraction of the pull wire will deploy and contract the distal assembly, respectively. The retraction mechanism may, as will be further described below, also include inflating a balloon or sliding a sheath.

The distal end 130 of the tubular component 100 may also, or alternatively, include an exchange mechanism at the distal end 130. FIG. 4 illustrates an alternative embodiment of distal end 130 of the tubular component 100 that may be connected to distal assembly 132 for providing stimulation and may include distal port 118. Distal port 118 may be capable of delivering food and/or fluids to, or removing gases from the targeted region of the GI tract. Distal port 118 may deliver pharmalogical medication to enterically stimulate the GI tract, alone or in combination with other stimulation modalities. Delivery/removal port 116 of handle 112, as depicted in FIG. 3, may be fluidly connected to distal port 118. Distal port 118 of FIG. 4 is exemplary and the exchange mechanism of the present disclosure may utilize multiple ports or other passages for the delivery and/or removal.

The distal end 130 may include a stimulation mechanism. The stimulation mechanism may, alone or in combination, have the ability to mechanically, electrically, or enterically stimulate (via, e.g., pharmacologic agent or other suitable modality) the GI tract. These stimulation modalities may operate alone, or incorporated with other stimulation modalities. The stimulation may be delivered through distal assembly 132 as depicted in FIG. 4.

In some embodiments, the parameters for stimulation may be, for example, random, set by the user, or based on the parameter sensed by the sensing mechanism (e.g. motion, stress, strain, contact impedance, electrical signals, and/or chemical biomarkers). These parameters may be induced in unique patterns to optimize the GI response. The stimulation may also be configured to (or as a mere accidental side effect) interfere with pain in the GI reflexes that may be contributing to POI.

In one embodiment, the stimulation mechanism may be through controlled mechanical manipulation. The stimulation mechanism may produce mechanical forces within the lumen of the small intestine to further promote controlled contractile activity. Mechanical manipulation may invoke the stretch receptors in the GI tract, specifically the jejunum, to induce coordinated peristaltic waves.

In some embodiments, the stimulation mechanism may stimulate the GI tract electrically. For example, electrical stimulation may “reset” the electrical pacing system of the entire GI tract, or just a portion that is causing lack of coordinated bowel activity. In such embodiments, a distal assembly 132 at the distal end 130 may include an arrangement of one or more flexible electrodes. The electrodes may, for example, be individually placed on the length of insulated wire (e.g., the Constellation Catheter by Boston Scientific Corporation of Maple Grove, Minn.), may span the entire uninsulated portion of the wire, or may be integrated into a flexible mesh or conductive material in order to maximize contact with the interior wall 22 of the small intestine 2. The electrodes may all be stimulating electrodes or some may be grounding electrodes and some stimulating electrodes. The electrodes may all stimulate simultaneously or in a pre-determined fashion such as to promote a cascade effect on the next cluster of adjacent gastric pacemaker cells, e.g. inches away. The distance between electrodes may be between 0-5″ apart.

The distal assembly 132 connected to the distal end 130 of the tubular component 100 may be comprised of electrical stimulators, such as electrodes, arranged in numerous configurations. Exemplary configurations are depicted in FIGS. 5-8. The electrical stimulators may be in a collapsed state upon insertion into the patient and then be deployed upon reaching the targeted area within the GI tract. The electrical stimulators may be designed to physically touch or be in electrical communication with an interior surface of the target region of the GI tract and may provide stimulation through physical contact or an electric field. In an expanded/deployed state, the distal assembly 132 may be configured to anchor the medical device within the body. The electrical stimulators may be configured to deliver electrical impulses capable of stimulating soft tissue, such as nerves and muscle, in the target region of the GI tract in order to generate contractions.

FIGS. 5A and 5B depict an alternative configuration of distal assembly 132 of FIGS. 2 and 4, wherein assembly 532 has a balloon-like′ configuration. FIG. 5A depicts assembly 532 (connected to distal end 130 of tubing 120) in the collapsed state after insertion into small intestine 2. The mechanisms of deployment and/or retraction of the electrical stimulators on assembly 532 may be via an inflatable system to expand assembly 532. FIG. 5B depicts 532 in its deployed state, in which assembly 532 is inflated until the electrical stimulators are in physical contact or electric communication with the interior wall 22 of the small intestine 2 at least at points 534.

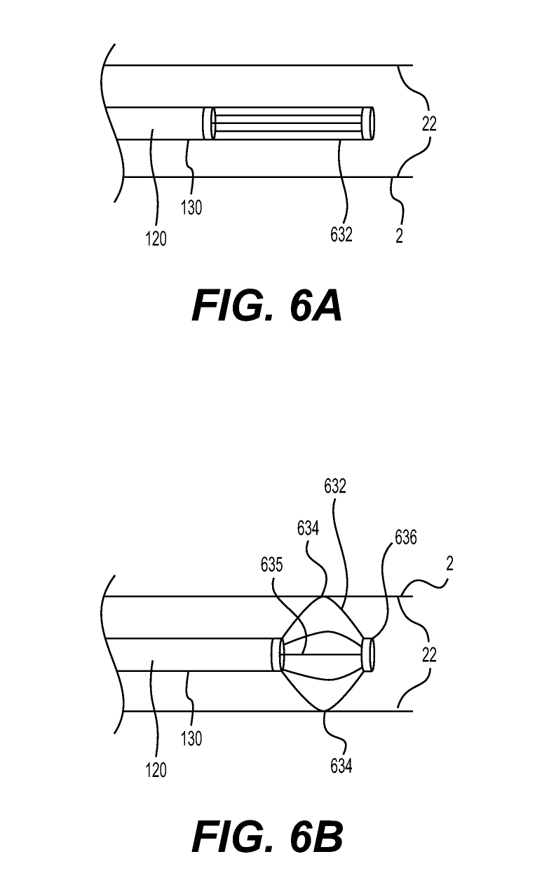

FIG. 6A illustrates assembly 632, connected to distal end 130 and after insertion into the small intestine 2. In FIG. 6A, assembly 632 is in its collapsed state. The mechanisms of deployment and/or retraction of the electrical stimulators on assembly 632 may be via a pull-wire or pulley system, wherein the distal end of assembly 632 is configured to be pulled toward proximal end 110. For example, a pull-wire 635 may be attached to a distal ring 636 and to the handle at the proximal end. Pulling pull-wire 635 retracts 636 to expand/deploy assembly 632. FIG. 6B depicts assembly 632 in its deployed state, in which the electrical stimulators of assembly 632 are in physical contact or electric communication with the interior wall 22 of the small intestine 2 at least at points 634.

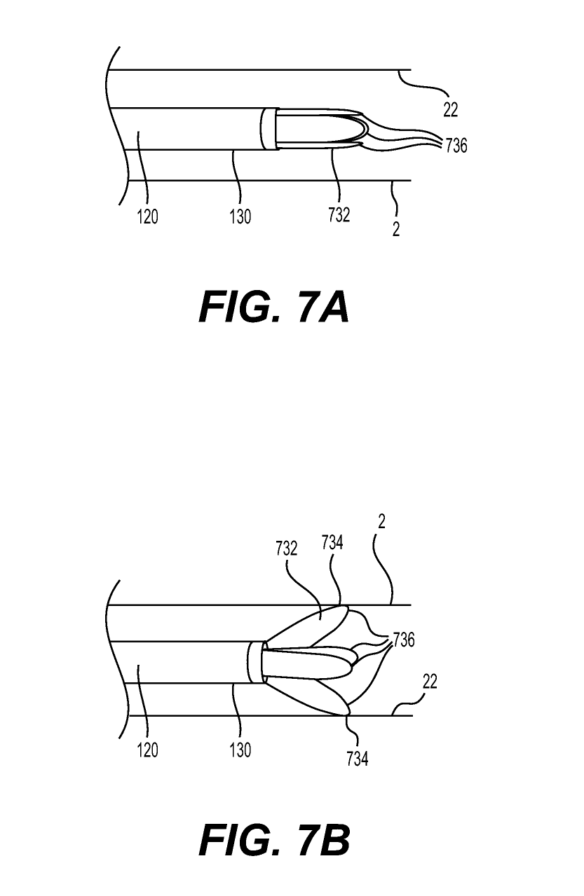

FIG. 7A depicts the collapsed state and FIG. 7B depicts the deployed state of an alternative embodiment of distal end 130 in which assembly 732 has a ‘flower petal’ configuration. The petals 736 of FIGS. 7A and 7B have rounded ends. The deployment mechanism may be a pull-wire or pulley mechanism whereupon the user pulls an attached wire that can deploy or retract the electrical stimulators. The deployment mechanism may additionally or alternatively employ a sheath to hold petals 736 of assembly 732 in a collapsed position until assembly 732 is pushed distally or the sheath is pulled proximally, thus allowing petals 736 of assembly 734 to transition to their natural, expanded state. The petals 736 may be configured to expand until the electrical stimulators of petals 736 are in physical contact and/or electrical communication with interior surface 22 of small intestine 2 at least at points 734.

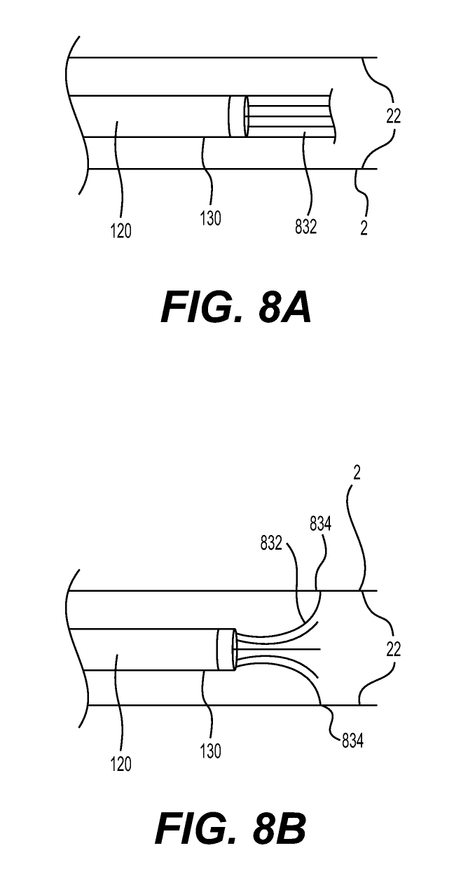

FIGS. 8A and 8B depict a ‘brush-like’ electrical stimulator configuration whereupon when the electrical stimulators are deployed they bend outwardly, like bristles of a paintbrush. The deployment mechanism may be, but is not limited to, a pull-wire or pulley mechanism whereupon the user pulls an attached wire that can deploy or retract the electrical stimulators. Additionally or alternatively, a deployment sheath may cover and then uncover the stimulators to allow them to expand to a biased position. FIG. 8A illustrates assembly 832 in the collapsed state. FIG. 8B illustrates assembly 832 in the deployed state, wherein the electrical stimulators may contact interior wall 22 of small intestine 2 at least at points 834.

The disclosed distal assemblies are merely exemplary and distal assembly 132 and the electrodes thereon may be configured in any arrangement. Even though the assemblies in the examples above contact the interior walls of the small intestine, the stimulation mechanism may be configured to stimulate any portion of the GI tract.

In another embodiment, stimulation through a pharmacologic agent may be utilized alone or incorporated into the electrical and/or mechanical stimulation mechanisms. In one embodiment, stimulation through a pharmacologic agent may include an enteric coating around the mechanical and/or electrical stimulators. In another embodiment, pharmacologic agents may be released through distal port 118 located at the distal end of the tubular component 100, as depicted in FIG. 4.

B. Control unit

The disclosed medical device may also include a control unit, as depicted in FIG. 9, capable of interfacing with handle portion 112 of the tubular component 100 to provide electricity to the electrical stimulators, to deliver food through the food port 116a, or expel gas through the gas port 116b from the targeted region of the GI tract depending upon desired usage of the device by the user. The control unit 900 may be powered by an external source such as an electrical outlet. In addition to those features shown, the control unit 900 may include buttons, knobs, touchscreens, or other user interfaces to control the sensing mechanism. The control unit 900 may be housed in the handle itself or in a separate apparatus.

The control unit 900 may be configured to enable the user to set patterns of electrical stimulation or may have pre-set electrical stimulation patterns. For example, the voltage may range 0.5 to 1.5V. In one embodiment, an electrical stimulation pattern may include a repeating stimulation pattern of 1V, 4 mA for 5 seconds, then 55 seconds off until resolution of POI.

In another example, the data from the sensing mechanism may be integrated to optimally control the stimulations to influence the contractions and/or relaxations in the intestinal smooth muscle cells. In an additional example, the pattern of electrical stimulation may be sequenced proximal to distal or vice versa. The electrical stimulation may be staggered or with the stimulators and grounds at varying locations on the electrodes. For example, the distal end of the stimulator would stimulate first and then, e.g., 0.5 seconds (or any suitable predetermined time) later the proximal end of the stimulator would stimulate. Additionally or alternatively, like with the Constellation Catheter, the electrodes located most distally and proximally may be the stimulators and then the middle electrodes would serve as the grounds. Control 910 may be configured to turn the device on and off, or may be used to set or select patterns, including those referenced above.

C. Methods for Operation

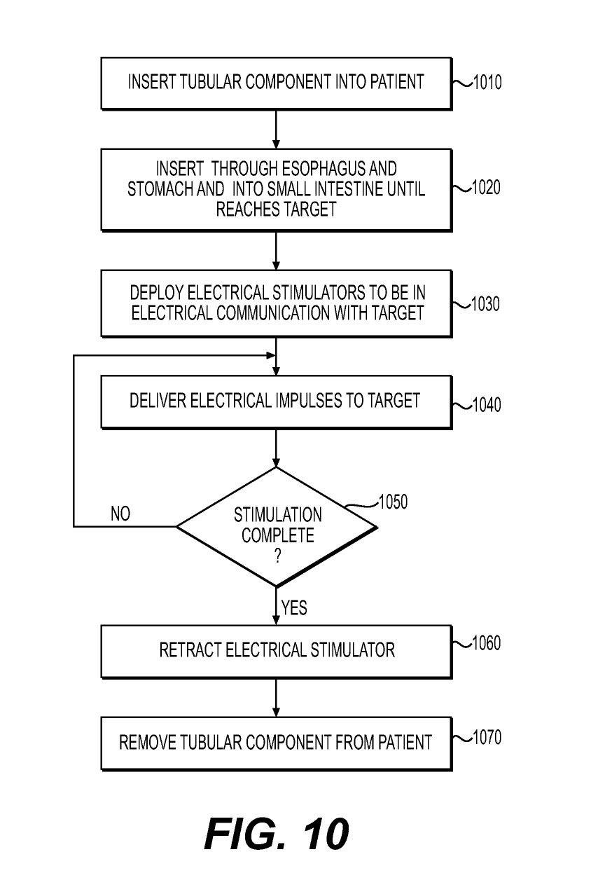

FIG. 10 illustrates an exemplary method of use of a medical device for treatment of POI. As shown in step 1010, at least a portion of the tubular component of the medical device may be inserted into the nose or mouth of a patient's body. In step 1020, the tubular component may be further inserted through the esophagus, stomach, and into the small intestine until it reaches the target region. The target region may be, for example, the duodenum or the jejunal region of the small intestine. Once the device is at the target region, the user may deploy electrically stimulating components, such as electrodes, to be in electrical communication with the target region of the GI tract (step 1030). In some embodiments, the stimulators may be electrical, mechanical, or enteric, or a combination thereof. In step 1040, the electrical stimulator may deliver electrical impulses to the target region. The stimulation may be complete after, for example, a predetermined amount of time, once the user determines, or once a parameter is observed that indicates the stimulation is a success (such as a motion within the GI tract that indicates sustained coordinated contractions have commenced). Stimulation may not be complete as defined by step 1050, until several rounds of electrical stimulation have been applied. For example, one round of electrical stimulation lasting approximately 5 hours may be applied to the target region. The device would remain within the GI tract at the target region while a healthcare practitioner or the control unit interpret the parameters sensed by the sensing mechanism. If sustained coordinated contractions are sensed during the break from electrical stimulation, the device may be removed. Alternatively, if sustained coordinated contractions are not sensed or are not within desired parameters, additional rounds of electrical impulses may be applied at the target region. The user may additionally choose to move the device into a more distal region of the GI tract, and apply additional electrical impulses at the new target region. This could be repeated and the device may remain in the patient, cycling through a program for, e.g., 16-24 hours or more before moving to step 1060. Once the stimulation is complete (as determined in step 1050), the electrical stimulator may be retracted into a collapsed position, in step 1060. After retraction of the electrical stimulator, the tubular component may be removed from the patient's body.

In some embodiments, if the user determines that the stimulation is unsuccessful, before step 1050, the user may retract the electrical stimulator, reposition, redeploy, and begin to deliver electrical impulses to a new target region. The steps may be repeated until the stimulation is determined complete (i.e. once sustained coordinated contractions of the GI tract begin).

In some embodiments, the target region may be selected based on the location of contractions in the GI tract. This location may be determined based on parameters sensed by the sensing mechanism, as described above.

In certain embodiments, the electrical impulses of step 1040 may be delivered in a pattern. This pattern may be pre-set, determined in real-time by the user, or these impulses may be actively controlled and optimized based on parameters collected from the sensing mechanism and implemented by the control unit, as described above. These parameters may include motion, stress, strain, contact impedance, electrical signals, and/or chemical biomarkers.

The many features and advantages of the disclosure are apparent from the detailed specification, and thus, it is intended by the appended claims to cover all such features and advantages of the disclosure which fall within the true spirit and scope of the disclosure. Further, since numerous modifications and variations will readily occur to those skilled in the art, it is not desired to limit the disclosure to the exact construction and operation illustrated and described, and accordingly, all suitable modifications and equivalents may be resorted to, falling within the scope of the disclosure.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical instrument, comprising:
an elongate component having a proximal end and a distal end, wherein the medical instrument is configured for insertion in a natural orifice of a patient and to traverse the gastrointestinal tract of the patient;
a handle at the proximal end; and
a stimulator at the distal end, wherein the stimulator is configured to stimulate the gastrointestinal tract to effect coordination of contractile peristaltic waves.

2. The medical instrument of claim 1, wherein the stimulator provides at least one of electrical, mechanical, and enteric stimulation.

3. The medical instrument of claim 1, wherein the stimulator is configured to be in a collapsed state for insertion into the gastrointestinal tract, and wherein the stimulator is configured to expand from the collapsed state to a deployed state to contact an interior surface of the gastrointestinal tract.

4. The medical instrument of claim 1, wherein the distal end of the elongate component includes at least one port for at least one of delivering food, delivering pharmacologic agents, removing waste, and removing gas.

5. The medical instrument of claim 1, further comprising a control unit configured to control one or more stimulation patterns of the stimulator based on at least one of real-time user input and pre-set stimulation patterns.

6. The medical instrument of claim 5, further comprising a sensor for sensing at least one parameter of motion, stress, strain, contact impedance, electrical signals, and chemical biomarkers, wherein the control unit is configured to adjust stimulation provided by the stimulator based on the least one parameter sensed by the sensor.

7. The medical instrument of claim 6, wherein the distal end is positioned based on the at least one parameter.

8. The medical instrument of claim 1, wherein the stimulator is configured to stimulate the gastrointestinal tract in a pattern of repeating stimulation, wherein the pattern comprises a stimulation of between 0.5V and 1.5V, until sustained coordinated peristaltic waves are sensed.

9. A medical instrument, comprising:
an elongate component having a proximal end and a distal end, wherein the medical instrument is configured for insertion in a natural orifice of a patient and to traverse the gastrointestinal tract of the patient;
a handle at the proximal end; and
a stimulator at the distal end, wherein the stimulator is configured to stimulate the gastrointestinal tract to effect coordination of contractile peristaltic waves.

10. The medical instrument of claim 9, wherein the stimulator provides at least one of electrical, mechanical, and enteric stimulation.

11. The medical instrument of claim 9, wherein the stimulator is configured to be in a collapsed state for insertion into the gastrointestinal tract, and wherein the stimulator is configured to expand from the collapsed state to a deployed state to contact an interior surface of the gastrointestinal tract.

12. The medical instrument of claim 9, wherein the distal end of the elongate component includes at least one port for at least one of delivering food, delivering pharmacologic agents, removing waste, and removing gas.

13. The medical instrument of claim 9, further comprising a control unit configured to control one or more stimulation patterns of the stimulator based on at least one of real-time user input and pre-set stimulation patterns.

14. The medical instrument of claim 13, further comprising a sensor for sensing at least one parameter of motion, stress, strain, contact impedance, electrical signals, and chemical biomarkers, wherein the control unit is configured to adjust stimulation provided by the stimulator based on the least one parameter sensed by the sensor.

15. A method for treatment of a gastrointestinal tract, comprising:
positioning a distal portion of a medical instrument, including a stimulator configured to stimulate tissue, at a target region within the gastrointestinal tract; and
stimulating tissue of the target region, via the stimulator, to effect coordination of peristaltic waves.

16. The method of claim 15, further comprising producing patterns of stimulation based on at least one of real-time user input and pre-set stimulation patterns.

17. The method of claim 15, further comprising deploying the stimulator to be in physical or electrical communication with tissue of the target region.

18. The method of claim 15, wherein stimulating tissue includes at least one of electrical, mechanical, and enteric stimulation.

19. The method of claim 15, further comprising sensing a location of a contraction and selecting the target region based on the location of the contraction.

20. The method of claim 19, further comprising sensing a parameter within the patient's body and basing a pattern for stimulating the target region on the sensed parameter, and repeating the pattern for stimulation until sustained coordinated peristaltic waves are sensed.
